(19) United States
(12) Patent Application Publication
(10) Pub. No.: US 2017/0105857 A1
(43) Pub. Date: Apr. 20, 2017

(54) RETRIEVAL OF MEDICAL DEVICES

(71) Applicant: Covidien LP, Mansfield, MA (US)

(72) Inventors: Frank SCARPINE, Brea, CA (US); Francis BERNARD, Dana Point, CA (US); Maria De Jesus SANSON, San Clemente, CA (US)

(21) Appl. No.: 14/885,848

(22) Filed: Oct. 16, 2015

(51) Int. Cl. A61F 2/95

(52) U.S. Cl. CPC A61F 2/95; A61F 2002/9528; A61F 2002/9511; A61F 2002/011

(57) ABSTRACT

A medical device retrieval system can include an elongate core member and an expandable member coupled to a distal portion of the elongate core member. The expandable member can move between collapsed and expanded states. The expandable member has an exterior having barbs positioned therealong. The expandable member can be expanded to contact and engage a portion of a medical device positioned within the vasculature of a patient in order to retrieve and remove the medical device therefrom. When the expandable member moves to the collapsed state, it can move the medical device from a higher profile configuration to a lower profile configuration to facilitate retrieval.

### BACKGROUND

### Field of the Inventions

The present disclosure generally relates to a system and method for retrieving a medical device within a vessel, more particularly, it relates to a system and method for retrieving an endoluminal therapeutic device within the vasculature of a patient.

### Description of the Related Art

Intravascular stents are well known in the medical arts for the treatment of vascular stenoses or aneurysms. Stents are prostheses that expand radially or otherwise within a vessel or lumen to provide support against the collapse of the vessel. Methods for delivering these intravascular stents are also well known.

In conventional methods of introducing a compressed stent into a vessel and positioning it within in an area of stenosis or an aneurysm, a guiding catheter having a distal tip is percutaneously introduced into the vascular system of a patient. The guiding catheter is advanced within the vessel until its distal tip is proximate the stenosis or aneurysm. A guidewire positioned within an inner lumen of a second, inner catheter and the inner catheter are advanced through the distal end of the guiding catheter. The guidewire is then advanced out of the distal end of the guiding catheter into the vessel until the distal portion of the guidewire carrying the compressed stent is positioned at the point of the lesion within the vessel. Once the compressed stent is located at the lesion, the stent may be released and expanded so that it supports the vessel.

The final position of the stent within the vessel is generally determined by its initial placement or landing within the vessel. In some situations, the stent may initially be “landed” in a suboptimal location within the vessel. Using traditional methods and apparatuses, it may be very difficult for a clinician to reposition the stent within the vessel. For example, a clinician may be unable to recapture, collapse, withdraw, or resheath the stent back into the catheter after the stent has been partially expanded within the vessel. Further, even if properly placed in a vessel, some situations may arise in which a stent must be retrieved.

### SUMMARY

At least one aspect of the disclosure provides methods and apparatuses for retrieving an intravascular device or devices (e.g., stent or stents) in the body. The retrieving device can be inserted into the vasculature and be passed to the location in the vasculature where the intravascular device is positioned. The retrieving device can be used with a variety of intravascular device. For example, in some embodiments, the retrieving device can be inserted into a lumen of an intravascular stent and engage the stent from within the lumen to cause the stent to collapse in diameter. Additionally, such a retrieving device can engage with the stent to facilitate recapturing of the stent to within a catheter.

The subject technology is illustrated, for example, according to various aspects described below. Various examples of aspects of the subject technology are described as numbered embodiments (1, 2, 3, etc.) for convenience. These are provided as examples and do not limit the subject technology. It is noted that any of the dependent embodiments may be combined in any combination with each other or one or more other independent embodiments, to form an independent embodiment. The other embodiments can be presented in a similar manner. The following is a non-limiting summary of some embodiments presented herein:

Clause 1. A medical device retrieval system, comprising: an elongate core member comprising a distal portion; an expandable member coupled to the distal portion, expandable member being movable between collapsed and expanded states to cause an exterior of the expandable member to contact an inner surface of a medical device when the expandable member is radially expanded within the medical device; and a plurality of extensible members coupled to the exterior of the expandable member, the extensible members being movable to engage the medical device when the expandable member moves to the expanded state, the extensible members being configured to exert a collapsing force on the medical device when (i) the extensible members are engaged with the medical device and (ii) the expandable member is moved to the collapsed state to thereby reduce an outer profile of the medical device and facilitate recapturing the medical device.

Clause 2. The system of Clause 1, wherein the plurality of extensible members comprise hook and loop fasteners.

Clause 3. The system of any of the preceding Clauses, wherein the plurality of extensible members extend along an entirety of the exterior of the expandable member.

Clause 4. The system of any of the preceding Clauses, wherein the plurality of extensible members comprise arcuate protrusions, each protrusion coupled to the expandable member at an acute angle relative to the exterior of the medical device.

Clause 5. The system of Clause 4, wherein in the collapsed state, the exterior of the expandable member has a cylindrical shape.

Clause 6. The system of any of Clauses 4-5, wherein in the expanded state, the exterior of the expandable member has a bulbous shape.

Clause 7. The system of any of Clauses 4-5, wherein in the expanded state, the exterior of the expandable member has a cylindrical shape.

Clause 8. The system of any of the preceding Clauses, further comprising a catheter having a lumen, the core member being longitudinally movable within the lumen between an extended position and a retracted position to facilitate recapturing the medical device within the lumen.

Clause 9. The system of Clause 8, wherein the catheter comprises a split end portion, the split end portion being expandable from a first diameter to a second diameter to facilitate recapturing the medical device within the lumen.

Clause 10. The system of Clause 9, wherein the split end portion comprises at least one longitudinally extending cut separating the split end portion into a plurality of movable sections, the plurality of movable sections being movable to permit the split end portion to expand from the first diameter to the second diameter.

Clause 11. A medical device retrieval system, comprising: an elongate core member comprising a distal portion; and an expandable member coupled to the distal portion, the expandable member being movable between collapsed and expanded states, the expandable member comprising an exterior having barbs positioned therealong, wherein in the expanded state, spaces open between the barbs and the exterior of the expandable member, and in the collapsed state, the spaces between the barbs and the exterior are closed to engage portions of a medical device within the closed spaces, the expandable member being movable to the collapsed state to force the medical device from a higher profile configuration to a lower profile configuration.

Clause 12. The system of Clause 11, wherein in the expanded state, the barbs extend in a direction away from the expandable member exterior.

Clause 13. The system of Clause 12, wherein in the expanded state, end portions of the barbs are spaced apart from the expandable member exterior at a first distance, and in the collapsed state, the end portions are spaced apart from the expandable member exterior at a second distance, the second distance being less than the first distance.

Clause 14. The system of any of Clauses 11-13, wherein the barbs comprise hook and loop fasteners.

Clause 15. The system of any of Clauses 11-14, wherein the barbs extend along an entirety of the exterior of the expandable member.

Clause 16. The system of any of Clauses 11-15, wherein the barbs comprise arcuate protrusions, each protrusion coupled to the expandable member at an acute angle relative to the exterior of the medical device.

Clause 17. The system of any of Clauses 11-16, wherein in the collapsed state, the exterior of the expandable member has a cylindrical shape.

Clause 18. The system of any of Clauses 11-17, wherein in the expanded state, the exterior of the expandable member has a bulbous shape.

Clause 19. The system of any of Clauses 11-17, wherein in the expanded state, the exterior of the expandable member has a cylindrical shape.

Clause 20. The system of any of Clauses 11-19, further comprising a catheter having a lumen, the core member being longitudinally movable within the lumen between an extended position and a retracted position to facilitate recapturing the medical device within the lumen.

Clause 21. The system of Clause 20, wherein the catheter comprises a split end portion, the split end portion being expandable from a first diameter to a second diameter to facilitate recapturing the medical device within the lumen.

Clause 22. The system of Clause 21, wherein the split end portion comprises at least one longitudinally extending cut separating the split end portion into a plurality of movable sections, the plurality of movable sections being movable to permit the split end portion to expand from the first diameter to the second diameter.

Clause 23. A method of retrieving a medical device, the method comprising: inserting a retrieval system into a lumen of a patient, the system comprising an elongate core member, an expandable member coupled to the core member, and a plurality of extensible members coupled to the expandable member; advancing the expandable member adjacent to the medical device, the medical device being in an expanded configuration; radially expanding the expandable member to contact the plurality of extensible members against the medical device and engaged the expandable member with the medical device; and radially collapsing the expandable member to move the medical device from the expanded configuration to a collapsed configuration.

Clause 24. The method of Clause 23, wherein the medical device comprises a tubular member, and wherein the advancing the expandable member adjacent to the medical device comprises inserting the expandable member into a lumen of the tubular member.

Clause 25. The method of any of Clauses 23-24, wherein the radially expanding comprises inflating the expandable member.

Clause 26. The method of any of Clauses 23-24, wherein the radially expanding comprises urging opposing ends of the expandable member toward each other to longitudinally foreshorten the expandable member.

Clause 27. The method of Clause 26, wherein the urging opposing ends of the expandable member toward each other comprises maintaining a longitudinal position of a first of the opposing ends while moving a second of the opposing ends relative to the first opposing end.

Clause 28. The method of Clause 27, wherein the elongate core member comprises first and second portions coupled to the first and second opposing ends, respectively.

Clause 29. The method of Clause 28, wherein the first portion comprises a hollow tubular member having a lumen, and wherein the second portion comprises an elongate member extending within the lumen of the first portion.

Clause 30. The method of any of Clause 23-29, further comprising proximally retracting the medical device, in the collapsed configuration, into a catheter.

Clause 31. The method of Clause 30, wherein the catheter comprises a split end portion having first and second tubular sections and a mouth diameter, and the proximally retracting comprises deflecting the tubular sections to increase the mouth diameter, thereby facilitating retrieval of the medical device into the catheter.

Clause 32. The method of any of Clauses 23-31, wherein before radially collapsing the expandable member, the method further comprises longitudinally shifting the expandable member against the medical device to urge the extensible members into engagement with the medical device.

Clause 33. The method of Clause 32, wherein the longitudinally shifting comprises proximally retracting the expandable member relative to the medical device to urge the extensible members into engagement with the medical device.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

The accompanying drawings, which are included to provide further understanding of the subject technology and are incorporated in and constitute a part of this specification, illustrate aspects of the disclosure and together with the description serve to explain the principles of the subject technology.

FIG. 1 is a perspective view of a medical device retrieval system, according to some embodiments.

FIGS. 2-6 are side, cross-sectional views of the medical device retrieval system shown in FIG. 1 being used to retrieve a medical device, according to some embodiments.

FIGS. 7A-8 are enlarged side, cross-sectional views of protrusions on a medical device retrieval system, according to some embodiments.

FIGS. 9-10 are side views of medical device retrieval systems having expandable members, according to some embodiments.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

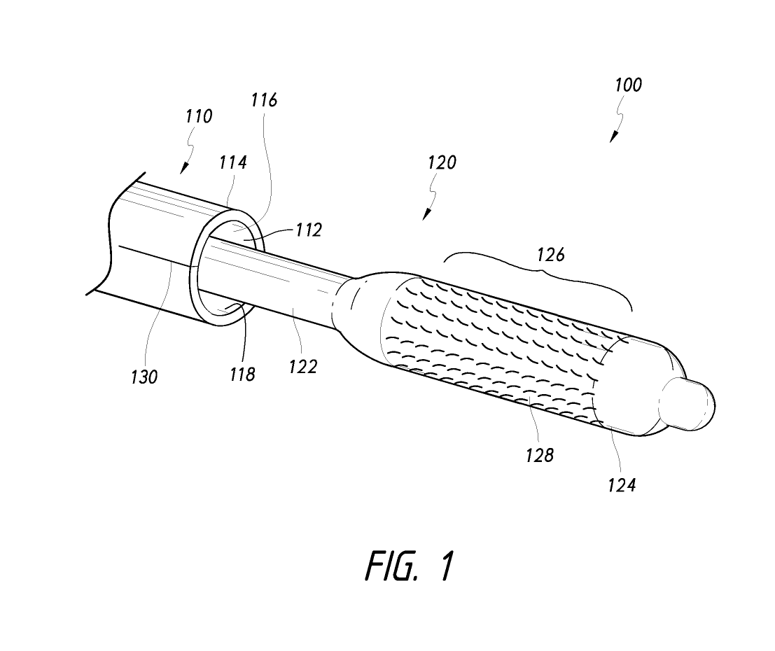

FIGS. 1-10 depict embodiments of a medical device retrieval system 100 which may be used to retrieve, reposition, recapture, and/or collect at least a portion of a medical device, such as but not limited to a stent 200, into a hollow anatomical structure such as a blood vessel 102. The retrieval system 100 can comprise an elongate tube or catheter 110 which slidably receives a core assembly 120 configured to move through the catheter 110. In use, the retrieval system 100 can be inserted into the vasculature of a patient over a guide wire until reaching a target area where a medical device resides. In order to retrieve the medical device, the retrieval system 100 can contact and engage the medical device. In some embodiments, the device can be forced to a lower profile state by the system 100 and retrieved from the vasculature.

As noted, the medical device can be in the form of the stent, other generally tubular medical device(s) or other such interventional medical devices, such as a vascular plug or occluder, a filter, a valve, a stent-graft, or an anchor. The stent can comprise a braided stent or other form of stent such as a laser-cut stent, roll-up stent, etc. The stent can optionally be configured to act as a “flow diverter” device for treatment of aneurysms, such as those found in blood vessels including arteries in the brain or within the cranium, or in other locations in the body such as peripheral arteries. The stent can optionally be similar to any of the versions or sizes of the PIPELINE™ Embolization Device marketed by Medtronic of Minneapolis, Minn. USA. The stent can further alternatively comprise any suitable tubular medical device and/or other features.

FIG. 1 illustrates the core assembly 120 extending distally out of the catheter 110. The depicted catheter 110 (see FIGS. 1-6) has a proximal end portion (not shown) and an opposing distal end portion 114, which can be positioned at a treatment site within a patient, an internal lumen 116 extending from the proximal end portion to the distal end portion 114, and an inner surface 118 facing and defining the lumen 116. At the distal end portion 114, the catheter 110 has a distal opening 112 through which the core assembly 120 may be advanced beyond the distal end portion 114. The proximal end portion may include a catheter hub. The catheter 110 can define a generally longitudinal axis extending between the proximal end portion and the distal end portion 114. When the retrieval system 100 is in use, the longitudinal axis need not be straight along some or any of its length.

The catheter 110 can optionally comprise a microcatheter. For example, the catheter 110 can optionally comprise any of the various lengths of the MARKSMAN™ catheter available from Medtronic of Minneapolis, Minn. USA. The catheter 110 can optionally comprise a microcatheter having an inner diameter of about 0.030 inches or less, and/or an outer diameter of 3 French or less near the distal end portion 114. Instead of or in addition to these specifications, the catheter 110 can comprise a microcatheter which is configured to percutaneously access the internal carotid artery, or a location within the neurovasculature distal of the internal carotid artery, via its distal opening 112.

Information regarding additional embodiments of the catheter 110, and additional details and components that can optionally be used or implemented in the embodiments of the catheter described herein, can be found in U.S. Patent Application Publication No. US 2011/0238041 A1, published on Sep. 29, 2011, titled Variable Flexibility Catheter. The entirety of the aforementioned publication is hereby incorporated by reference herein and made a part of this specification.

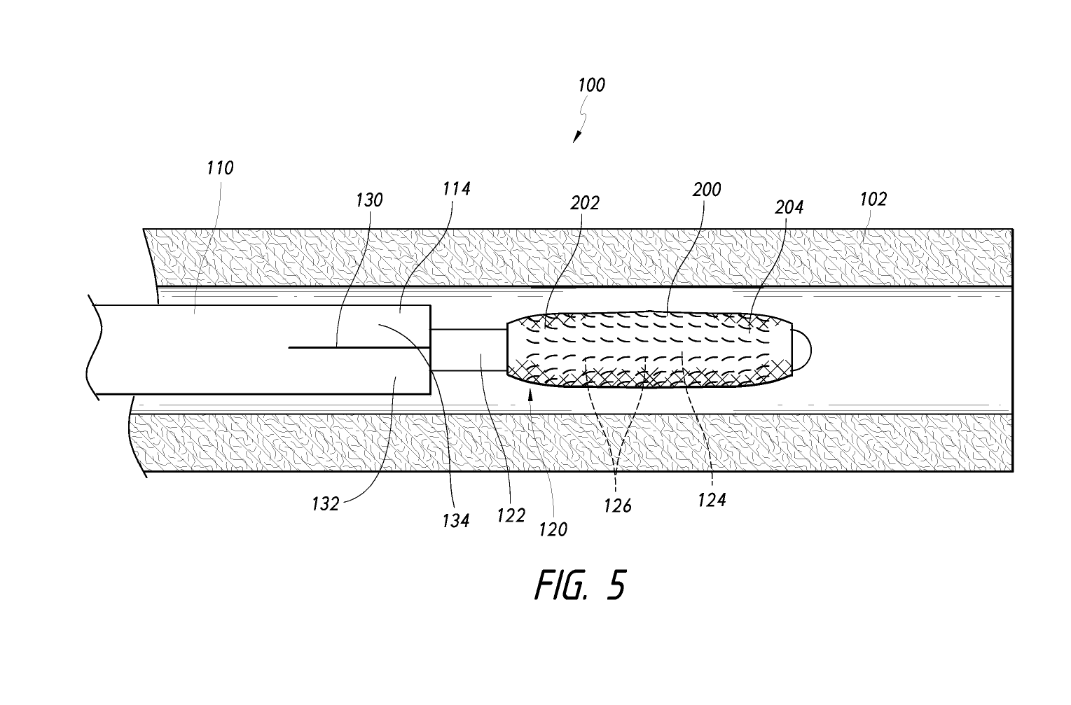

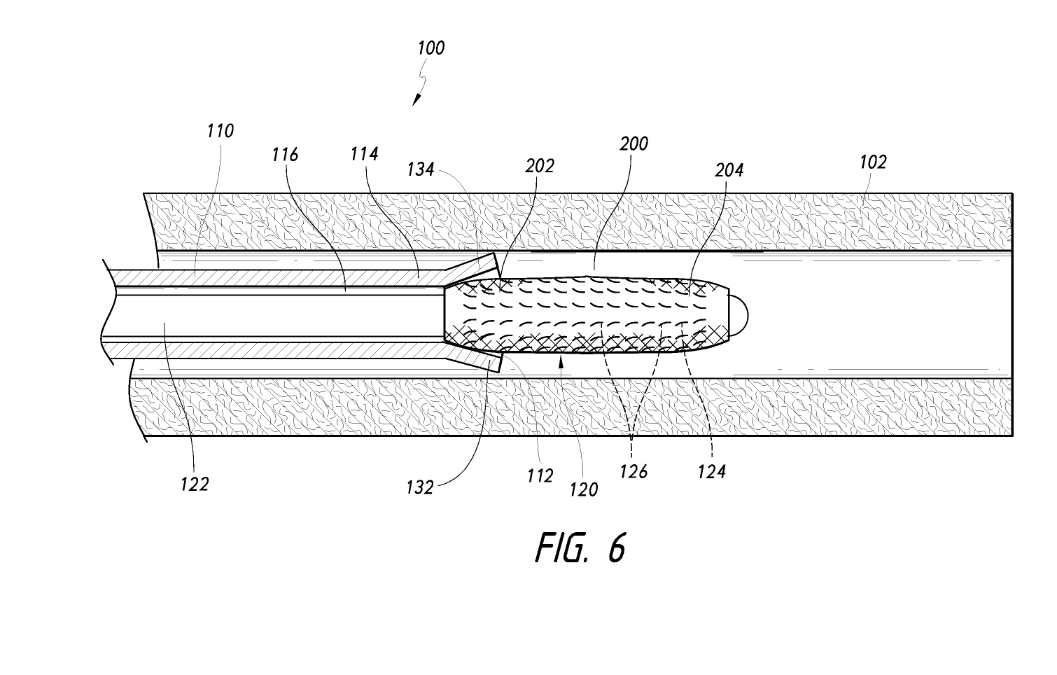

In accordance with some embodiments, the catheter 110 can also comprise at least one longitudinal slit 130. The longitudinal slit 130 can extend proximally from the distal end portion 114 to divide the distal end portion 114. For example, in some embodiments, the distal end portion 114 can comprise one or more sections that can be radially separated in order to increase the diameter or size of the distal opening 112. As discussed further below, and as shown in FIGS. 5 and 6, at least two sections 132, 134 can be urged apart from each other in order to permit recapture of a medical device within the lumen 116.

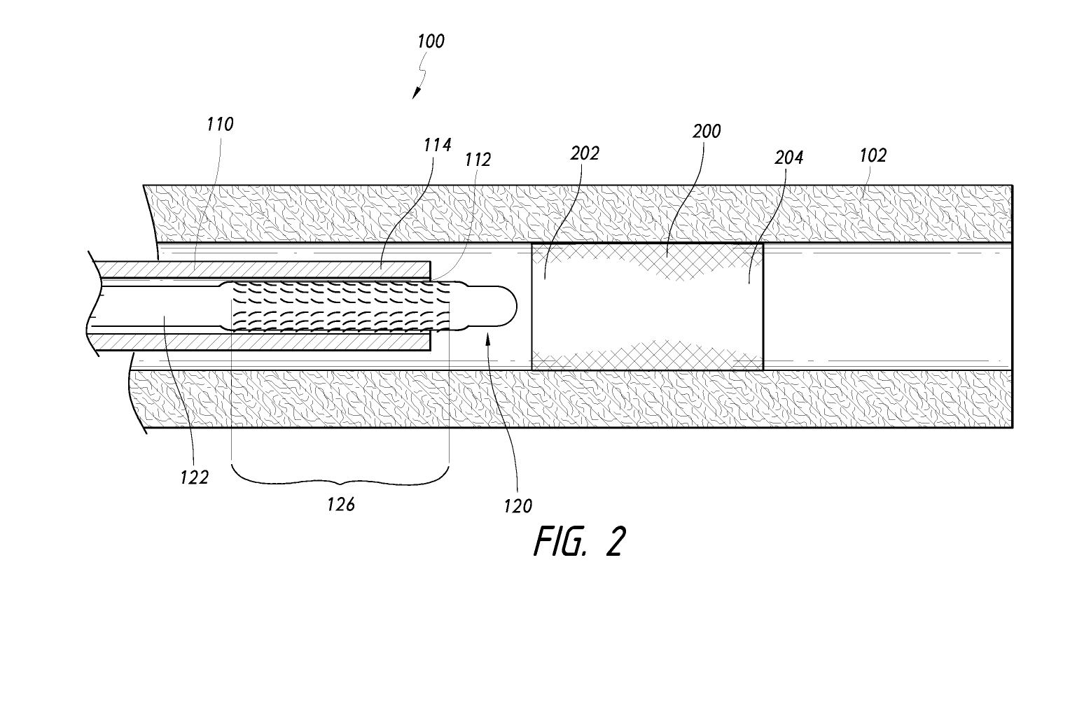

Referring still to FIG. 1, the core assembly 120 can comprise a core member 122 and an expandable member 124 coupled to a distal portion of the core member 122. The expandable member 124 can be actuated to selectively move between an expanded state and a collapsed state. In use, a clinician can selectively actuate the expandable member 124 in order to cause the expandable member 124 to engage a medical device that has been implanted into a patient's vasculature, as discussed below with respect to FIGS. 2-6.

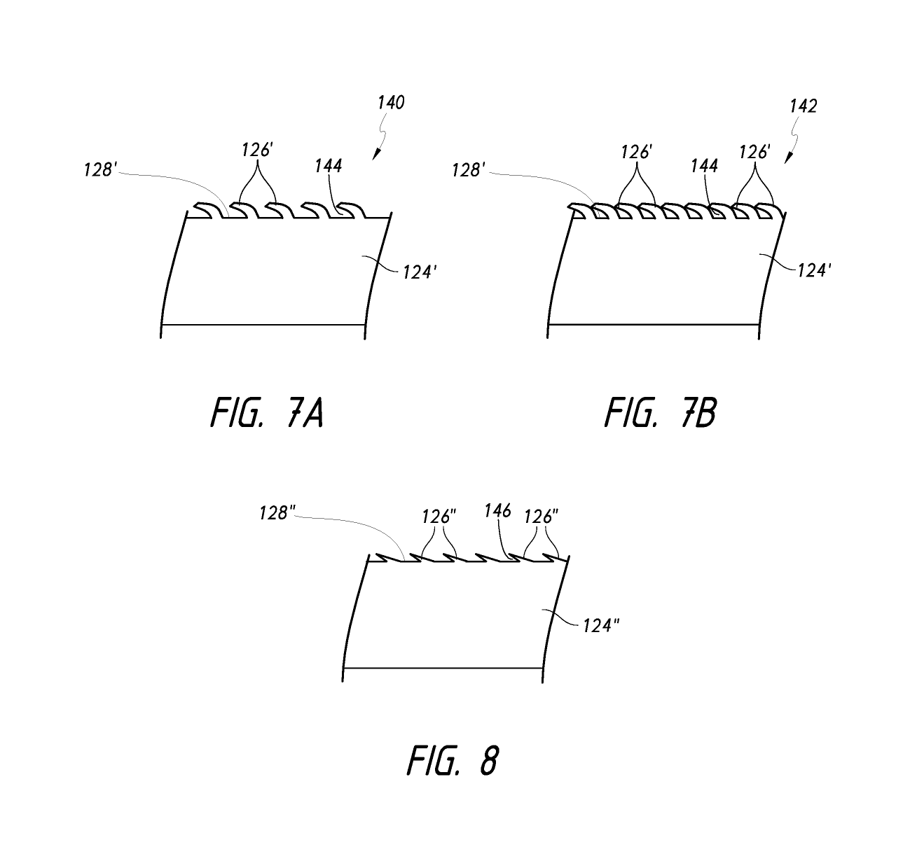

In order to facilitate engagement between the expandable member 124 and the medical device, the expandable member 124 can comprise a plurality of extensible members or protrusions 126. The extensible members 126 can extend from an exterior surface 128 of the expandable member 124. The extensible members 126 can extend along a portion or the entirety of the longitudinal extent of the expandable member 124. Further, the extensible members 126 can extend around a portion or the entirety of the circumference of the expandable member 124. In some embodiments, the extensible members 126 can move to engage the medical device based on the expanded or collapsed state of the expandable member 124. For example, the extensible members 126 can be engaged with the medical device when the expandable member 124 is in the expanded state and contacts the medical device. Further, the expandable member 124 can be moved to the collapsed state, causing the extensible members 126 to exert an inward-pulling and/or collapsing force on the medical device, thereby reducing an outer profile of the medical device and facilitate recapturing the medical device. Some embodiments of extensible members are discussed further below with regard to FIGS. 7A-8.

In accordance with some embodiments, the expandable member 124 and the extensible members 126 can be formed from the same material. However, the expandable member 124 and the extensible members 126 can also be formed from separate materials and coupled or attached to each other. For example, the expandable member 124 can comprise a relatively soft material to facilitate relatively high-friction or “high-grip” contact with a portion (e.g., an inner surface) of the medical device and/or facilitate expansion and elasticity of the expandable member 124. The expandable member 124 and the extensible members 126 can differ in mechanical properties such as hardness. The extensible members 126 can be formed from a relatively hard or stiff material to facilitate low-friction engagement with the portion of the medical device.

In some embodiments, the expandable member 124 can be formed from a relatively soft polymer or elastomer such as silicone, rubber (e.g., Chronoprene™), thermoplastic polyurethane (e.g., Tecoflex™), polyethylene terephthalate (PET), or polyether block amide (e.g., Pebax™). Whether made of such materials, or of other materials, the expandable member 124 can have a durometer of between 10A and 50A, or between 15A and 40A, or about 20A, or about 25A. The expandable member 124 can comprise a variety of three-dimensional shapes. For example, the expandable member 124 can comprise a cylindrical shape, a rounded-end cylindrical shape, a barrel shape, a shape that tapers in either or both directions, and/or a bulbous shape, and can comprise one or more drawdowns in diameter to create two or more bulges, which can have equal or differing diameters or sizes.

Further, instead of or in addition to the above-recited materials and/or properties of the expandable member 124, in some embodiments, the extensible members 126 can be formed from polyimide, e.g., a polyimide tube; alternatively a tubular metallic coil (e.g., a stainless steel coil) could be employed, stamped metal strips having a plurality of tabs extending from the strip, or a metal tube, either with or without slots or a spiral cut formed in the sidewall. Whether made of such materials, or of other materials, the extensible members 126 can have a higher durometer than the expandable member 124, e.g., above 70D or between 70D and 100D.

In some embodiments, the expandable member 124 and the extensible members 126 can be integrally formed. For example, both layers could be formed from a single cylinder of soft material. Further, in some embodiments, the material can be processed to make its outer layer of extensible members 126 harder, softer, and/or have a coefficient higher-friction than the exterior of the expandable member 124.

The expandable member 124 can comprise a length that is about equal to the length of the medical device to be retrieved. For example, the expandable member 124 can have a length of at least 7 mm, 8 mm, 9 mm, 10 mm, 11 mm, 12 mm, 13 mm, 15 mm, 18 mm, 20 mm, 23 mm, 25 mm, 28 mm, 30 mm, 33 mm, 35 mm, 37 mm, 40 mm, 43 mm, 45 mm, 48 mm, 50 mm, 53 mm, 55 mm, 58 mm, or 60 mm. In some embodiments, for example where the retrieval system is to be used in narrow and/tortuous vasculature, such as the neurovasculature, the expandable member 124 can be of relatively small outside diameter (e.g., 0.040″ or less, or 0.030″ or less, or 0.027″ or less, or about 0.020″).

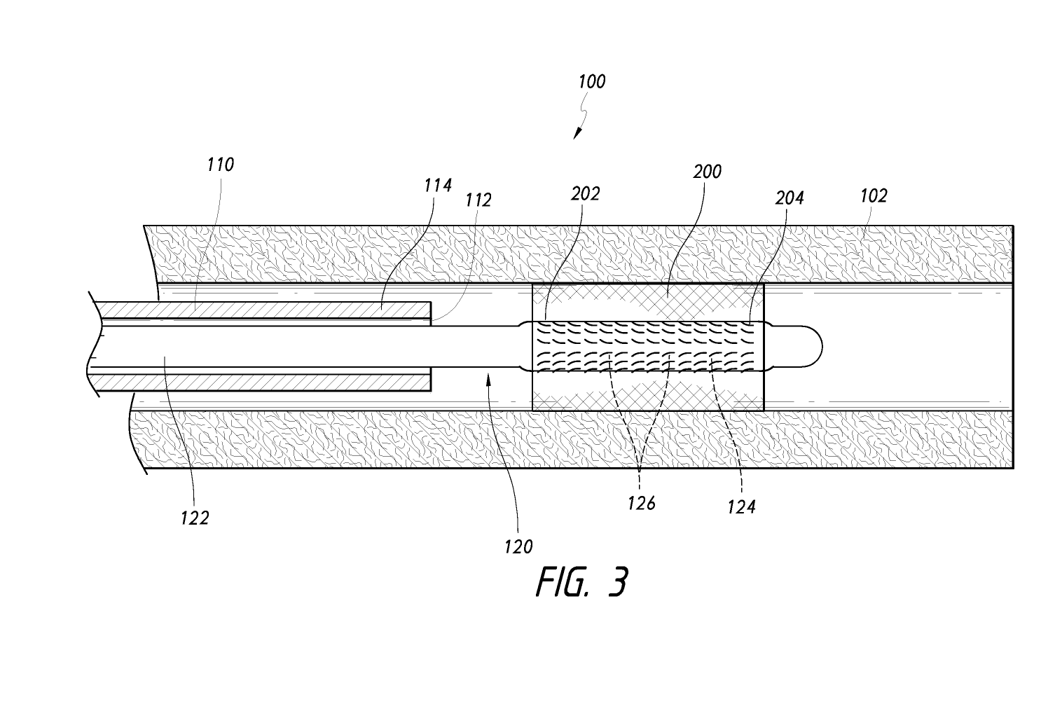

Referring now to FIGS. 2-6, an embodiment of a method of retrieving a medical device is illustrated. FIG. 2 illustrates the retrieval system 100 positioned adjacent to a medical device (illustrated as but not limited to stent 200) within a blood vessel 102 of a patient. The stent 200 may need to be removed for one of a variety of reasons, and may have a patent lumen that permits the retrieval system to be positioned at least partially within the stent 200. For example, as shown in FIG. 3, after the distal end portion 114 of the retrieval system 100 is positioned adjacent to the stent 200, the core assembly 120 can be distally advanced out of the distal opening 112 of the catheter 110. In the illustrated embodiment, the expandable member 124 of the core assembly 120 can be inserted into a lumen of the stent 200. However, in some embodiments where the medical device does not have a patent lumen (e.g., or the medical device is a sheet of material, a misshapen stent, a flattened filter, or other non-tubular structure), the expandable member 124 can be positioned so as to longitudinally overlap at least a portion of the medical device within the blood vessel 102.

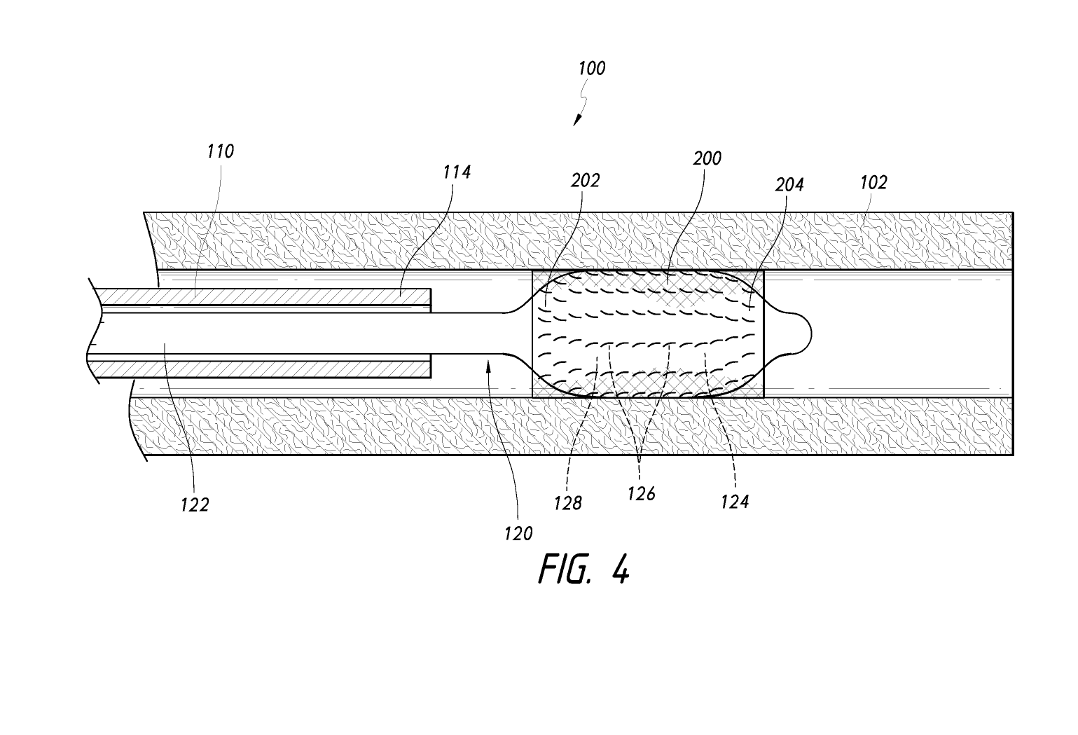

Once in position, as shown in FIG. 4, the expandable member 124 can be expanded into contact with at least a portion of the stent 200. When expanded, the exterior 128 of the expandable member 124 can be pressed against an inner profile or inner surfaces of the stent 200. As the expandable member 124 moves to its expanded state, the extensible members 126 can contact the inner surfaces of the stent 200 and be urged through apertures, pores, or windows of the stent 200.

The degree to which the extensible members 126 become enmeshed with the stent 200 affects the amount or proportion of the surface area of the expandable member 124 (and extensible members 126) that contacts the stent 200 to create an engagement between the expandable member 124 and the stent 200. Further, in accordance with some embodiments, the extensible members 126 can radially interlock with portions of the stent 200 in order to create the engagement.

For example, in some embodiments, after expanding the expandable member 124, the extensible members 126 can be further enmeshed or interlocked with portions of the stent 200 by exerting a longitudinal force on the core assembly 120. The longitudinal force can be a distal or proximal force that causes the expandable member 124 to shift or slide, or otherwise move, along the interior or inner surface of the stent 200. In accordance with some embodiments, this slight shift can cause the extensible members 126 to increase the degree to which they are engaged with portions of the stent 200 (especially those extensible members 126 that extend at an acute angle relative to the exterior 128 of the expandable member 124, thereby forming a pocket between the exterior 128 and the extensible member 126).

After the expandable member 124 is engaged with the stent 200, the expandable member 124 can be collapsed, thereby exerting an inward pulling and/or radially collapsing force on the stent 200. In some embodiments, when a balloon is used for the expandable member 124, a negative inflation pressure can be created using a syringe, which can exert a radially inward force on the stent 200. However, in some embodiments, the expandable member 124 can comprise a shape-set or shape memory structure that is biased to a collapsed state. Thus, an expansive force can be exerted on the shape memory structure, to urge it to an expanded state, and thereafter, the expansive force can be removed, thereby allowing the structure to return to its default collapsed state. Accordingly, in any of the embodiments of the expandable member 124, the collapsing force exerted by the expandable member 124 can exceed the spring force or radially outward force exerted by the stent 200. As these forces are applied and the stent 200 contracts, the stent can achieve a contracted state as shown in FIG. 5.

Thereafter, as illustrated in FIG. 6, the expandable member 124 and the stent 200 can be proximally withdrawn into the catheter 110. In some embodiments, the distal opening 112 of the catheter 110 can readily receive the combination of the expandable member 124 and stent 200, thereby allowing the stent 200 to be recaptured or withdrawn into the lumen 116 of the catheter 110.

However, some embodiments can be configured such that the catheter 110 comprises a compliant or expandable distal opening 112. For example, the distal end portion 114 can comprise a longitudinal slit or cut 130 that enables the distal opening 112 to have a variable or expandable diameter. Some embodiments can be configured such that a plurality of slits or cuts 130 divide the distal end portion 114 into two or more sections 132, 134, as shown in FIG. 6. Accordingly, as the proximal and 202 of the stent 200 begins to contact the distal end portion 114 of the catheter 110, continued proximal force of the core assembly 120 can cause the sections 132, 134 of the distal end portion 114 to spread apart, thereby facilitating ingress of the stent 200 engaged on the expandable member 124. As the expandable member 124 and the stent 200 are further drawn into the catheter lumen 116, the inner surface 118 of the catheter 110 can further drawdown the profile of the stent 200 until the stent 200 is fully received within the catheter lumen 116.

As noted above, the extensible members can comprise the protrusions that extend from the exterior of the expandable member. The protrusions can comprise barbs, hooks, thorns, hook and loop fasteners, surface roughness, coatings, or combinations thereof. For example, FIGS. 7A-8 illustrate extensible members that can be used in some embodiments.

For example, FIGS. 7A-7B illustrate cross-sections of an expandable member 124′ and its extensible members 126′ in an expanded state 140 (FIG. 7A) and in a collapsed state 142 (FIG. 7B). As shown, the extensible members 126′ can comprise a plurality of protrusions extending from an exterior 128′ of the expandable member 124′. The protrusions can comprise oblong shapes that extend at an acute angle relative to the exterior 128′. As shown in FIG. 7A, in some embodiments, the extensible members 126′ can each define a space 144 between themselves and the exterior 128′. During the engagement process, portions of the medical device can become enmeshed with the extensible members 126′ in these spaces 144. Further, while these spaces 144 can provide a radial boundary to a portion of the medical device received therein (e.g., a filament of a stent), some embodiments can optionally be configured such that the plurality of extensible members 126′ move from a spaced apart configuration (as shown in FIG. 7A) to a more closely bounded configuration (as shown in FIG. 7B). Thus, in FIG. 7B, the extensible members 126′ can provide a complete boundary to the spaces 144, closing the space 144 of a given extensible member 126′ using an adjacent extensible member 126′. Thus, although some embodiments can provide secure engagement using unbounded spaces 144, some embodiments can be implemented in which the spaces 144 are closed as the expandable member 124′ moves to the collapsed state.

FIG. 8 illustrates another embodiment of an expandable member 124″ having a plurality of extensible members 126″ that extend from the exterior 128″ of the expandable member 124″. In such an embodiment, the extensible members 126″ can similarly form spaces 146 between the extensible members 126″ and the exterior 128″. The extensible members 126″ represent yet another configuration of protrusions that can be used in accordance with some embodiments. When the expandable member 124″ is in the expanded state and in contact with the medical device, such extensible members 126″ can tend to permit movement of the expandable member 124″ relative to the medical device in one direction, but quickly engage with portions of the medical device when the expandable member 124″ is moved in the opposite direction.

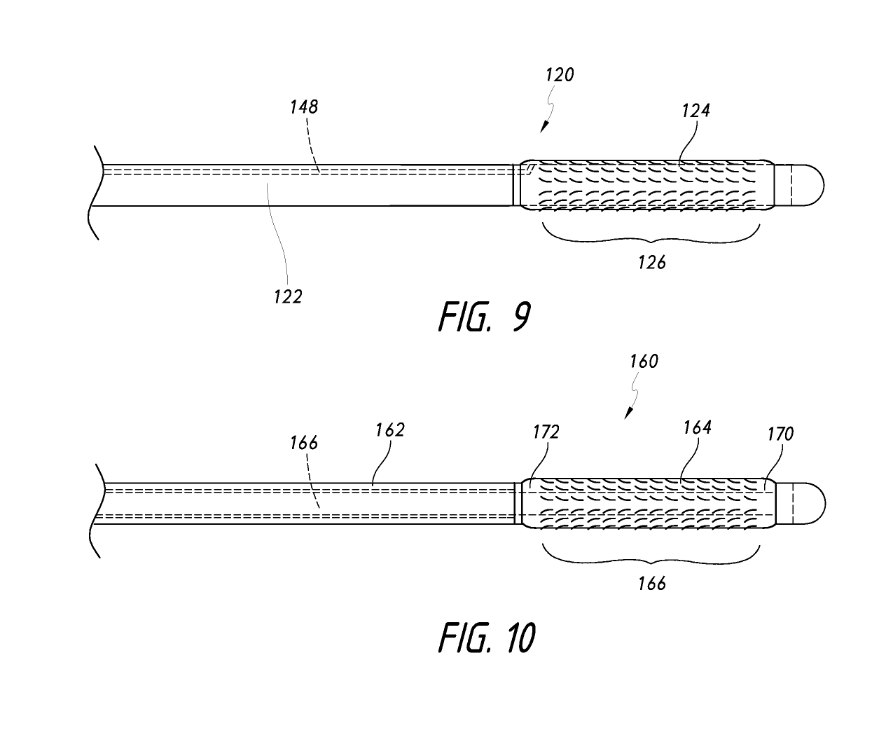

As illustrated in FIG. 9, the core assembly 120 can be configured such that the expandable member 124 comprises a balloon coupled to (e.g., mounted on) the distal end portion of the core member 122. The balloon can be kept deflated or otherwise collapsed against the core member 122 until it is desired to recapture or remove the medical device or otherwise move the medical device within the vasculature (e.g., repositioning the medical device within the vasculature). The balloon can be inflated via an inflation lumen 148 to engage the inner surface of the medical device, thereby gripping the medical device in cooperation with the extensible members 126. As noted above, upon so engaging or gripping the medical device, the balloon can be used to collapse the medical device. For example, a negative inflation pressure can be exerted, via a syringe, that will collapse the balloon and pull the medical device into a collapsed state. Thereafter, the core assembly 120 can retract the medical device back into the catheter by pulling the core member 122 proximally, in accordance with the re-sheathing methods described herein.

Further, FIG. 10 illustrates an embodiment of a core assembly 160 having a core member that comprises an outer actuation tube 162 and an inner actuation member 166 that can selectively move an expandable member 164 between collapsed and expanded states. As illustrated, the inner actuation member 166 can extend through a lumen of the outer actuation tube 162 and be coupled to a distal end portion 170 of the expandable member 164. The outer actuation tube 162 can be coupled to a proximal end portion 172 of the expandable member 164. In order to expand the expandable member 164, the distal and proximal end portions 170, 172 can be longitudinally drawn together by proximally retracting the inner actuation member 166 relative to the outer actuation member 162. As this occurs, the shape of the expandable member 164 can move towards a cylindrical or bulbous shape, as discussed with respect to some embodiments disclosed herein. Further, such actuation can allow the extensible members 166 to be deployed and engaged with the medical device.

In some embodiments, the expandable member 164 can comprise a shape-set or shape memory structure that is biased to a collapsed state. Thus, in order to move the expandable member 164 to the expanded state, an expansive force can be exerted on the shape memory structure by forcing the distal and proximal end portions 170, 172 toward each other. After the expandable member 164, has engaged the medical device, the expansive force can be removed (e.g., the clinician can cease to exert the expansive force), thereby allowing the structure to return to its default collapsed state. This movement of the expandable member 164 to its collapsed state can provide sufficient draw-down force on the medical device to cause the medical device to move to a collapsed state. Thereafter, the medical device and the battle member 164 can be withdrawn into the catheter.

Further, in accordance with some embodiments, a distal end of the core assembly can comprise a marker that facilitates visualization of the location of the core assembly and expandable member under fluoroscopy, for example. Other visualization techniques can be used, as well as other locations for markers. For example, the marker can be positioned at the distal end of the core member and/or at a proximal end of the expandable member.

In the present disclosure, numerous references are made to moving the catheter axially over the core assembly, and moving the core assembly axially within the catheter. Except where specifically noted to the contrary, all such references to one form of this relative movement should be understood to include the other as an alternative.

The apparatus and methods discussed herein are not limited to the retrieval of a medical device or stent within the vascular system but may include any number of further treatment applications. Other treatment sites may include areas or regions of the body including any hollow anatomical structures.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A medical device retrieval system, comprising:
an elongate core member comprising a distal portion;
an expandable member coupled to the distal portion, expandable member being movable between collapsed and expanded states to cause an exterior of the expandable member to contact an inner surface of a medical device when the expandable member is radially expanded within the medical device; and
a plurality of extensible members coupled to the exterior of the expandable member, the extensible members being movable to engage the medical device when the expandable member moves to the expanded state, the extensible members being configured to exert a collapsing force on the medical device when (i) the extensible members are engaged with the medical device and (ii) the expandable member is moved to the collapsed state to thereby reduce an outer profile of the medical device and facilitate recapturing the medical device.

2. The system of claim 1, wherein the plurality of extensible members comprise hook and loop fasteners.

3. The system of claim 1, wherein the plurality of extensible members extend along the exterior of the expandable member.

4. The system of claim 1, wherein the plurality of extensible members comprise arcuate protrusions, each protrusion coupled to the expandable member at an acute angle relative to the exterior of the medical device.

5. The system of claim 4, wherein in the collapsed state, the exterior of the expandable member has a cylindrical shape.

6. The system of claim 4, wherein in the expanded state, the exterior of the expandable member has a bulbous shape.

7. The system of claim 4, wherein in the expanded state, the exterior of the expandable member has a cylindrical shape.

8. The system of claim 1, further comprising a catheter having a lumen, the core member being longitudinally movable within the lumen between an extended position and a retracted position to facilitate recapturing the medical device within the lumen.

9. The system of claim 8, wherein the catheter comprises a split end portion, the split end portion being expandable from a first diameter to a second diameter to facilitate recapturing the medical device within the lumen.

10. The system of claim 9, wherein the split end portion comprises at least one longitudinally extending cut separating the split end portion into a plurality of movable sections, the plurality of movable sections being movable to permit the split end portion to expand from the first diameter to the second diameter.

11. A medical device retrieval system, comprising:
an elongate core member comprising a distal portion; and
an expandable member coupled to the distal portion, the expandable member being movable between collapsed and expanded states, the expandable member comprising an exterior having barbs positioned therealong, wherein in the expanded state, spaces open between the barbs and the exterior of the expandable member, and in the collapsed state, the spaces between the barbs and the exterior are closed to engage portions of a medical device within the closed spaces, the expandable member being movable to the collapsed state to force the medical device from a higher profile configuration to a lower profile configuration.

12. The system of claim 11, wherein in the expanded state, the barbs extend in a direction away from the expandable member exterior.

13. The system of claim 12, wherein in the expanded state, end portions of the barbs are spaced apart from the expandable member exterior at a first distance, and in the collapsed state, the end portions are spaced apart from the expandable member exterior at a second distance, the second distance being less than the first distance.

14. The system of claim 11, wherein the barbs comprise arcuate protrusions, each protrusion coupled to the expandable member at an acute angle relative to the exterior of the medical device.

15. A method of retrieving a medical device, the method comprising:
inserting a retrieval system into a lumen of a patient, the system comprising an elongate core member, an expandable member coupled to the core member, and a plurality of extensible members coupled to the expandable member;
advancing the expandable member adjacent to the medical device, the medical device being in an expanded configuration;
radially expanding the expandable member to contact the plurality of extensible members against the medical device and engaged the expandable member with the medical device; and
radially collapsing the expandable member to move the medical device from the expanded configuration to a collapsed configuration.

16. The method of claim 15, wherein the medical device comprises a tubular member, and wherein the advancing the expandable member adjacent to the medical device comprises inserting the expandable member into a lumen of the tubular member.

17. The method of claim 15, wherein the radially expanding comprises inflating the expandable member.

18. The method of claim 15, wherein the radially expanding comprises urging opposing ends of the expandable member toward each other to longitudinally foreshorten the expandable member.

19. The method of claim 15, further comprising proximally retracting the medical device, in the collapsed configuration, into a catheter.

20. The method of claim 19, wherein the catheter comprises a split end portion having first and second tubular sections and a mouth diameter, and the proximally retracting comprises deflecting the tubular sections to increase the mouth diameter, thereby facilitating retrieval of the medical device into the catheter.
